(19) United States
(12) Patent Application Publication
(10) Pub. No.: US 2004/0122399 A1
(43) Pub. Date: Jun. 24, 2004

(54) Package for discretely carrying interlabial and other feminine care products

(75) Inventor: Mary L. McDaniel, Appleton, WI (US)

Correspondence Address: DORITY & MANNING, P.A., POST OFFICE BOX 1449, GREENVILLE, SC, 29602-1449, US

(73) Assignee: KIMBERLY-CLARK WORLDWIDE, INC.

(21) Appl. No.: 10/323,708

(22) Filed: Dec. 18, 2002

(51) Int. Cl. A61F 13/15; A61F 13/20

(52) U.S. Cl. 604/385.02

(57) ABSTRACT

An individually wrapped feminine care absorbent article package is provided to provide consumers with the ability to discretely carry individual feminine care articles. An absorbent article is contained in a wrapper material configured into a closed pouch, the absorbent article being removed by a consumer opening the pouch and withdrawing the article. An attachment mechanism is disposed directly on an outer surface of the pouch and provides a consumer with the ability to discretely attach the pouch to their person or clothing so as to be not outwardly visible yet readily accessible to the consumer.

### FIELD OF THE INVENTION

The present invention relates to the field of feminine hygiene products, and more particularly to the packaging of such products.

### BACKGROUND

A broad range and wide variety of absorbent articles configured for absorption of bodily exudates such as menstrual fluid are well known. With respect to feminine hygiene, the art has offered two basic types of feminine hygiene protection: namely sanitary napkins, and panty liners developed for external wear about the pudendal region, and tampons, developed for residence within the vaginal cavity and accordingly for interruption of menstrual flow therefrom prior to such menstrual flow reaching the vestibule. Hybrid feminine hygiene protection devices, attempting to merge the structural features of both sanitary napkins and tampons in a single type of device, have also been proposed, but have not seen a meaningful measure of acceptance due in part to perceived structural and anatomically functional disadvantages. Other less intrusive devices, known as labial or interlabial devices or pads and characterized by residing primarily within the wearer's vestibule while having a portion which at least partially resides external of the wearer's vestibule, have also been proposed.

Since the various types of absorbent articles are typically carried in a woman's purse or pocket prior to use, it is a common advantageous practice to individually wrap each article to keep it clean and sanitary. Various suitable wrapping materials are know in the art for this purpose. For example, the article wrapper may consist of one or more layers of a thin sheet or film of thermoplastic material, such as polyethylene, which is folded around the absorbent article in a pouch or envelope-like configuration and then sealed by the use of heat and/or pressure, ultrasonics, or an adhesive to form an individual package. The pouches are designed to be opened by breaking or tearing the material at or adjacent a seal in order to remove the absorbent article. Conventional packages are also typically designed so that a soiled article can be wrapped up in the opened package for later disposal. Flushable and biodegradable pouches (as well as absorbent articles) are also being investigated in the art.

Most women value their personal privacy and prefer not to advertise to others that they are carrying or using feminine care products. For some individuals, the “public” use of such products can be an anxious and often traumatic experience. Consumers often complain about the lack of discretion involved with carrying absorbent articles to the restroom, or locating such articles in a purse, carrying bag, etc. Unfortunately, conventional wrapping and packaging systems may only exacerbate this problem. Often, the exercise of locating the article in a purse or carry bag is “noisy” and difficult to disguise from those around an individual. The articles may be wrapped or packaged in materials having vibrant colors or other distinguishing features that are easily recognized by others. Not all consumers welcome a manufacturer's efforts to increase product recognition and awareness.

Thus, a need exists in the art to provide consumers with discrete means to disguise the fact that feminine care absorbent articles are being carried or used by an individual.

### SUMMARY

Objects and advantages of the invention will be set forth in part in the following description, or may be obvious from the description, or may be learned through practice of the invention.

The present invention relates to a unique wrapper or package for individually wrapped absorbent articles. Although the invention is not limited to the type of absorbent article, it is particularly well suited for individually wrapped interlabial devices.

In accordance with the invention, an individually wrapped feminine care absorbent article package is provided. The package includes an absorbent article, such as an interlabial device, carried in a pouch. The pouch is formed from a wrapper material and may be sealed or closed. An opening flap may be provided that is grasped by the consumer to open the pouch. The invention is not limited by any particular type of wrapper material or pouch configuration. In one embodiment, the wrapper material may include an outwardly facing layer of soft and supple fibrous material, and an inner layer of a generally liquid impermeable film material. The fibrous material will tend to reduce noise generated by the wrapper material that would otherwise be present with all-film wrappers.

The package includes an attachment mechanism disposed directly on an outer surface of the pouch, for example on the back side of the pouch. The attachment mechanism may be any one of a number of devices and provides a consumer with the ability to discretely attach the pouch to their person or clothing at a location such that the pouch is not outwardly visible but is readily accessible to the consumer. For example, in one embodiment, the attachment mechanism may be a strip or other deposition of adhesive on an outside surface of the pouch. A release liner may be placed over the adhesive. A consumer may remove the release liner and attach the pouch at any discrete location on their person. The adhesive may be a skin compatible adhesive and the pouch may be attached directly to the consumer's skin, for example at the small of the back, on the abdomen, etc. The pouch may be removably adhered to an article of clothing, for example under the lapel of a jacket, in a sleeve cuff, etc. An adhesive strip is a versatile embodiment of the attachment mechanism and provides a wide variety of options to the consumer.

The ability to discretely “hide” the package of the present invention depends in large part on the size of the absorbent article and pouch. For this reason, the invention is particularly well suited for interlabial absorbent devices. Such devices are relatively small and thus render themselves to convenient discrete attachment in places or locations that would not be possible with larger absorbent articles such as pantiliners, pads, and the like. It should be appreciated that the invention is not limited to any particular type or style of interlabial device.

Various other types of attachment mechanisms may be configured with the pouch. For example, the attachment mechanism may be a mechanical device such as a clip, pin, or the like.

In another embodiment, the attachment mechanism may be a magnet or magnetic strip that would allow for attachment of the pouch to any metal carried by the consumer, such as behind a belt buckle, attached to a consumer electronic device, etc.

In another embodiment, a strip or patch of a conventional hook-type material may be disposed on at least one exterior surface of the pouch. The hook-type material may attach directly to certain types of fabric or articles of clothing, or to a discretely placed patch of compatible loop material.

In still another embodiment, the attachment mechanism may include a relatively simple loop formed on the pouch whereby the pouch can be carried by any article passing through the loop, for example a belt, bra strap, etc.

The attachment device may be any manner of ribbon or string-like member (chain, cord, elastic member, etc.) that allows the pouch to be hung from a piece of clothing worn by the consumer or an article carried by the consumer.

It is also within the scope and spirit of the invention to provide consumers with the ability to hide the absorbent article package in plain view. For example, the pouch may be disguised as a women's accessory item, notion, or apparel item typically worn in plain sight. The attachment mechanism provides the ability to attach the disguised pouch at any location.

Aspects of the invention will be described in greater detail below by reference to embodiments illustrated in the attached drawings.

### BRIEF DESCRIPTION OF THE DRAWINGS

FIG. 1 is a perspective view of an absorbent article package in accordance with the invention.

FIG. 2 is a back perspective view of an absorbent article package with an attachment mechanism in accordance with the invention.

FIGS. 3A and 3B are back perspective views of an absorbent article package with an attachment mechanism in accordance with the invention.

FIG. 4 is a back perspective view of an absorbent article package with an attachment mechanism in accordance with the invention.

FIG. 5 is a back perspective view of an absorbent article package with an attachment mechanism in accordance with the invention.

FIG. 6. is a back perspective view of an absorbent article package with an attachment mechanism in accordance with the invention.

FIG. 7 is a back perspective view of an absorbent article package with an attachment mechanism in accordance with the invention.

### DETAILED DESCRIPTION

Reference will now be made in detail to embodiments of the invention, one or more examples of which are illustrated in the drawings. Each embodiment and example are provided for purposes of explaining the invention, and are not meant as a limitation of the invention. For example, features illustrated or described as part of one embodiment may be used with another embodiment to yield still a further embodiment. It is intended that the present invention include these and other modifications and variations as come within the scope and spirit of the invention.

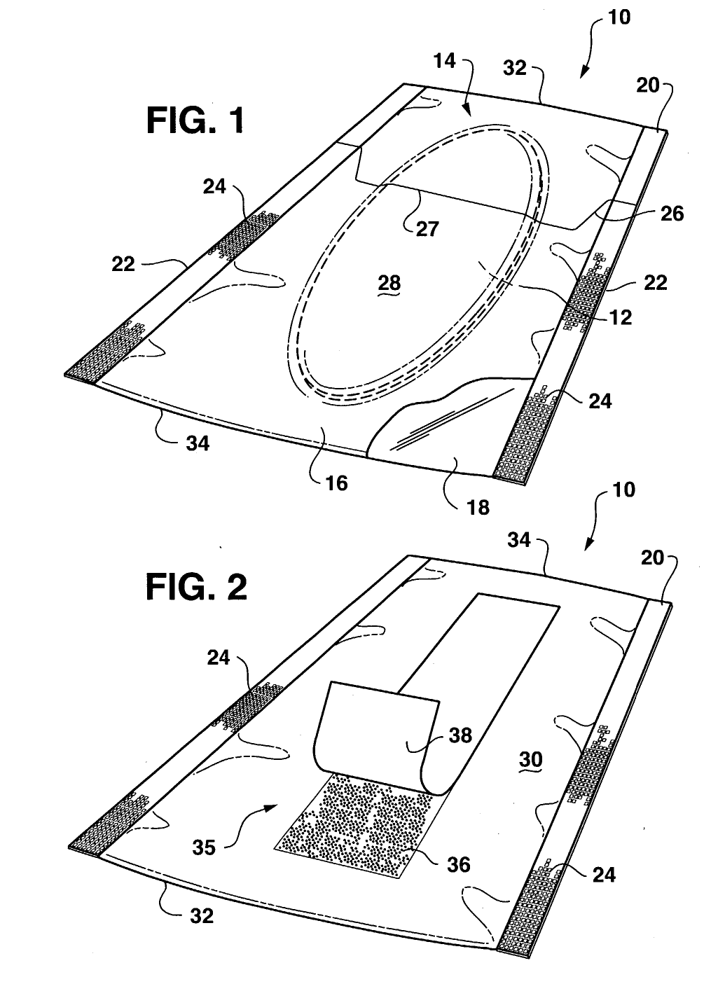

Various embodiments of an absorbent article package 10 in accordance with the invention are illustrated in the figures. The package 10 includes an individually wrapped absorbent article 12 contained therein. In FIG. 1, the article 12 is illustrated in dashed lines as a conventional interlabial device. It should be appreciated, however, that the article 12 may be any conventional feminine care absorbent article. The article 12 may be carried in the package 10 in a flat-out condition as illustrated, or in any folded configuration. The package 10 according to the invention is not limited by the type, style, or configuration of the absorbent article. Interlabial devices are relatively small and thin devices, and thus present many options for discretely disguising or hiding the package 10 that may not be available for packages containing larger absorbent articles, such as sanitary pads, liners, and the like. The construction of absorbent articles, including interlabial devices 12, are well known to those skilled in the art and need not be described in detail herein. Representative interlabial devices are described, for example, in U.S. Pat. Nos. 5,484,429; 3,726,277; 3,983,873; and 4,175,561, such references incorporated herein by reference for all purposes.

The package 10 includes a wrapper material 14 formed into a container or pouch 20. Typical pouch configurations in the art for storing individual absorbent articles 12 generally include a piece of liquid impermeable material, such as a film or the like, folded into an envelope-like pouch 20. Sides 22 are sealed or adhered together by conventional means along sealed portions 24. The pouch thus defines a back side 30, a front side 28, a top edge 32, and a bottom edge 34. The pouch 20 may also include a flap member 26 that provides a means for the consumer to conveniently open the pouch. The flap 26 may be a portion of the wrapper material folded over onto the front side 28, and may have sides that are sealed or adhered with the pouch sides 22, as generally illustrated in FIG. 1. The leading edge 27 of the flap 26 may be sealed across the front side 28, or left unsealed. One suitable pouch configuration is used by Kimberly-Clark Corp. of Neenah, Wisconsin, USA, for its Kotex® Ultrathin pads.

The wrapper material 14 may, in one embodiment, be formed primarily of any one of a number of thermoplastic film materials that is liquid impermeable, but vapor-permeable. One type of film that may be used is a nonporous, continuous film that, because of its molecular structure, is capable of forming a vapor-pervious barrier. Among the various polymeric films which fall into this category are films made from poly(vinyl alcohol), polyvinyl acetate, ethylene vinyl alcohol, polyurethane, ethylene methyl acrylate, and ethylene methyl acrylic acid to make them breathable. If desired, it is also possible to add fillers to the film such as, for example, calcium carbonate and titanium dioxide, to increase opacity, decrease cost, and create a breathable film upon subsequent stretching of the film.

Another type of film which may be useful is a microporous film. These films have a number of interconnecting voids or holes which provide pathways for the transportation of water molecules from one surface to another. The passageways are sufficiently small so that only vapors and not fluids can pass through.

In an alternate embodiment of the wrapper material 14, the material may include a soft fibrous material layer 16. This fibrous material 16 may be laminated to a film material 18 and be disposed outwardly facing, the film material 18 being inwardly facing and thus defining the interior of the pouch 20, as generally depicted in FIG. 1. The fibrous material layer 18 may comprise any one or combination of non-woven or woven materials and is intended to give the pouch a soft and cloth-like tactile feel, and to dampen noise associated with storing, carrying, and opening the pouch 20. Non-woven materials may be preferred from a manufacturing standpoint. Such non-woven materials and the process for their manufacture are well known to those skilled in the art. However, woven materials, including any manner of synthetic or natural cloth, are within the scope and spirit of the invention.

In an alternate embodiment, the wrapper material need not be a laminate material, and may comprise, for example, a single layer of non-woven material such as a spunbond layer.

The package 10 according to the invention includes an attachment mechanism, generally 35, provided on an exterior surface of the pouch 20. The attachment mechanism 35 may, for example, be provided on the back side 30 of the pouch 20, as illustrated in the figures. The attachment mechanism may take on various forms and includes any type of mechanical device or adhesive that allows a consumer to attach the pouch 20 to any desired discrete location on their person. The pouch 20 may, for example, be attached directly to skin, to an article of clothing, to a device such as a purse, briefcase, etc., carried or worn by the consumer, and so forth. The precise location or method by which a consumer chooses to disguise or hide the pouch 20 is not a limiting feature of the invention.

In one particular embodiment as illustrated in FIG. 2, the attachment mechanism 35 includes a patch, strip, or other deposition of an adhesive 36 applied directly to the back side 30 of the pouch 20. A release liner 38 may be disposed over the adhesive 36 to be removed by the consumer prior to attaching the pouch 20. The adhesive may be any conventional adhesive used to attach articles to clothing, and so forth. Many types of conventional feminine care products include a strip of adhesive disposed longitudinally along the outer cover to secure the article to an undergarment. Any one of these know adhesives may be used with the present invention.

In an alternative embodiment, the adhesive 36 may be a skin-compatible adhesive and thus provide the consumer with the ability to adhere the pouch 20 directly to their skin. For example, the pouch 20 may be adhered to the small of the back, to the abdomen, behind a leg, etc., and any other location on the body wherein the pouch 20 may be discretely adhered. Various skin-compatible adhesives are know in the art. Reference is made, for example, to U.S. Pat. Nos. 6,368,687; 6,316,524; and 5,658,270 (incorporated herein by reference) for a description of suitable skin-compatible adhesives.

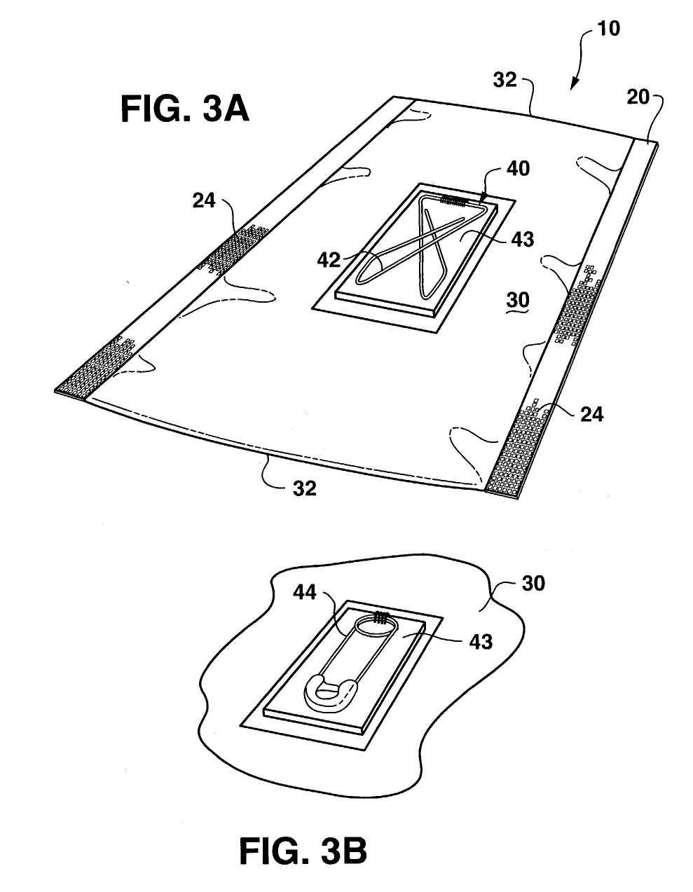

The attachment mechanism 35 may be any suitable mechanical device 40 that is affixed or configured directly on an exterior surface of the pouch 20, for example to the back side 30 of the pouch. The mechanical device should be relatively uncomplicated and inexpensive so as not to be cost or manufacturing prohibitive. Various mechanical attaching devices are illustrated in FIGS. 3 through 7.

Referring to FIG. 3A, one embodiment of the mechanical device 40 is a relatively simple clip mechanism. The mechanism may include a resilient tab or finger 42 formed on a back member 43. The entire device may be an inexpensive molded plastic component that is attached to the pouch 20. The tab 42, allows the pouch 20 to be attached to any piece of clothing or article that can slide between the tab 42 and back member 43. An alternate embodiment of a mechanical attachment device 40 is illustrated in FIG. 3B, and may include a pin 44 attached to a back member 43. It should be appreciated that any number of conventional mechanical devices may serve to readily attach the pouch 20 at a discrete location.

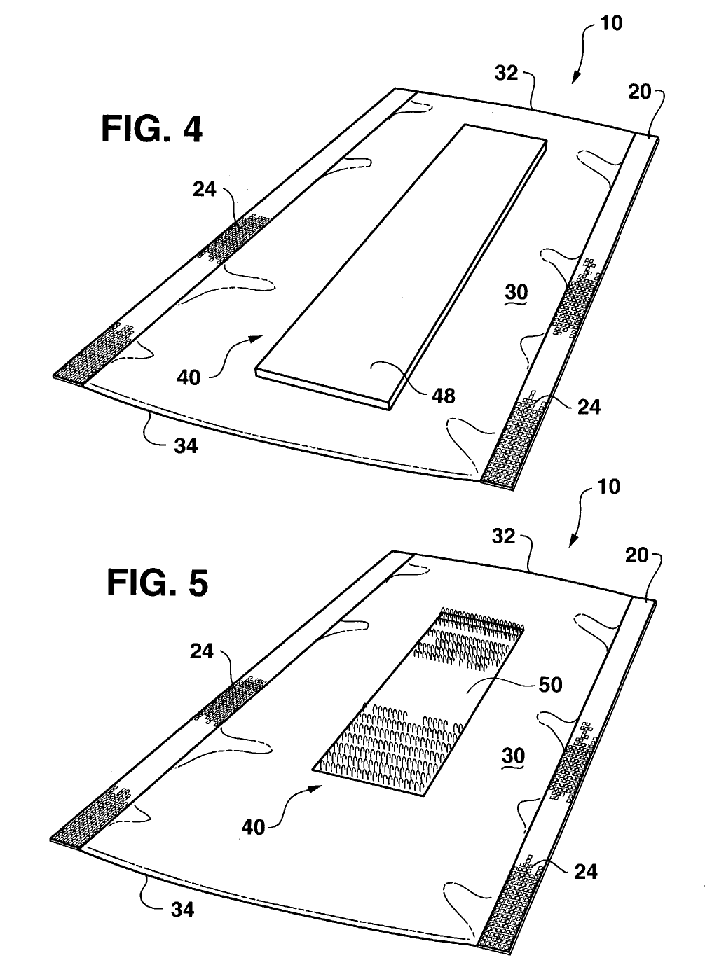

In an alternate embodiment illustrated in FIG. 4, the attachment mechanism may include a magnetic patch or strip 48 attached to a surface of the pouch 20. The magnetic strip 48 allows the consumer to attach the pouch 20 to any metallic component carried or worn by the consumer. For example, the pouch may be attached behind a belt buckle, behind a piece of jewelry, to the inside surface of a consumer electronic device such as a personal CD player, etc., to the inside of a zipper, and so forth.

In the embodiment of FIG. 5, the attachment mechanism is depicted as a patch of hook material 50. Such material is known to those skilled in the art. The hook material 50 may attach directly to certain fabrics, or to a complimentary piece of loop material that may be separately located by the consumer at a discrete location.

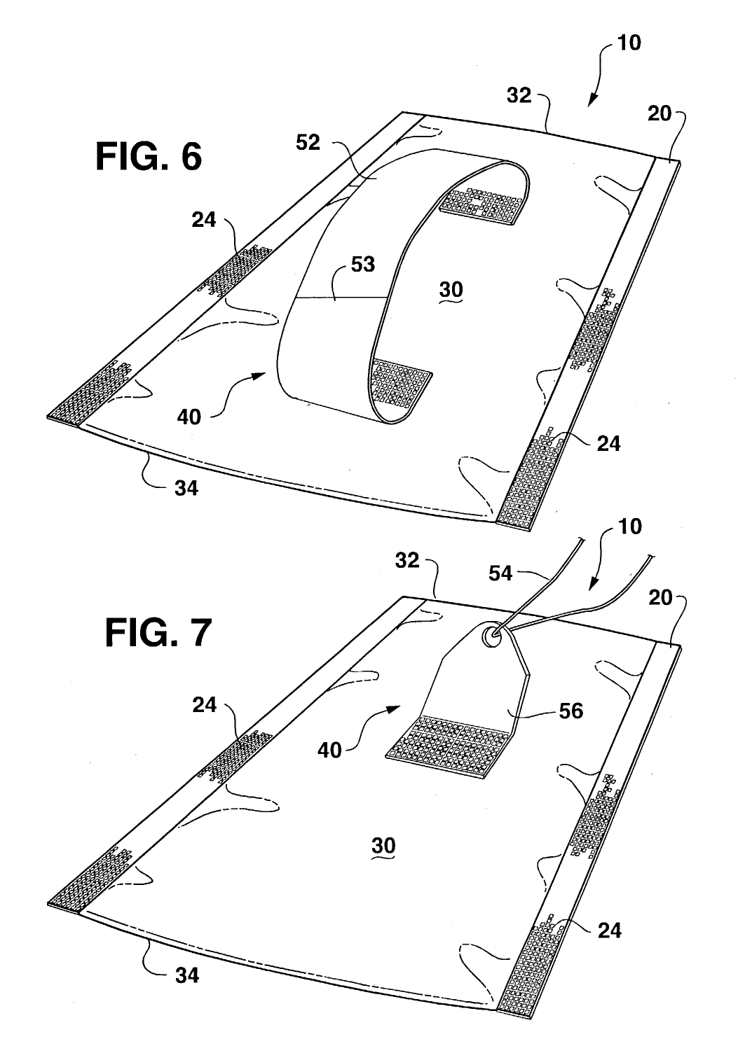

An alternative embodiment of the attachment mechanism is depicted in FIG. 6 as a simple loop 52 disposed on the back side 30 of the pouch. The loop 52 may be formed of the same wrapper material 14, or of another material and attached to the pouch 20. The loop 52 provides a device for the pouch to be carried by any type of strap, belt, and so forth. The loop 52 may be “broken” at a suitable location 53 so that a strap or other device may be inserted into or removed from the loop without being threaded through the loop. For example, a bra strap may be inserted into the loop at location 53.

In still a further embodiment illustrated in FIG. 7, the attachment mechanism includes any manner of string or ribbon device 54, including a cord, chain, elastic member, and so forth. The device 54 may be threaded through an eyelet structure 56 provided on an exterior surface of the pouch 20, and provides the consumer with a convenient device to hang or support the pouch 20 on any item carried or worn by the consumer.

It is also within the scope of the invention to disguise the appearance of the pouch 20 so that it may be attached and hidden “in plain view.” For example, the pouch may be disguised as a piece of jewelry, fashion apparel accessory item, notion, etc., and worn or attached on the body or clothing as such conventional devices typically are.

It should be appreciated by those skilled in the art that various modifications and variations may be made to the embodiments of the invention illustrated or described herein without departing from the scope and spirit of the invention as set forth in the appended claims and their equivalents.

What is claimed is:

1. An individually wrapped feminine care absorbent article package, comprising:
an absorbent article;
a wrapper material configured into a closed pouch, said absorbent article disposed within said pouch and removable by a consumer opening said pouch and removing said absorbent article;
an attachment mechanism disposed directly on an outer surface of said pouch, said attachment mechanism providing a consumer with the ability to discretely attach said pouch to their person or clothing so as to be not outwardly visible yet readily accessible to the consumer.

2. The absorbent article package as in claim 1, wherein said absorbent article comprises an interlabial article.

3. The absorbent article package as in claim 1, wherein said pouch comprises an envelope-type configuration defining an opening flap.

4. The absorbent article package as in claim 1, wherein said wrapper material comprises at least one fibrous material layer, said fibrous material layer outwardly disposed so as to define outer surfaces of said pouch.

5. The absorbent article package as in claim 4, wherein said wrapper material comprises a liquid impermeable layer inwardly disposed so as to define an interior of said pouch.

6. The absorbent article package as in claim 1, wherein said attachment mechanism comprises an adhesive disposed on at least one exterior surface of said pouch.

7. The absorbent article package as in claim 6, wherein said adhesive is a skin compatible adhesive.

8. The absorbent article package as in claim 6, further comprising a release liner attached to said adhesive.

9. The absorbent article package as in claim 1, wherein said attachment mechanism comprises a mechanical device.

10. The absorbent article package as in claim 9, wherein said mechanical device comprises a clip.

11. The absorbent article package as in claim 1, wherein said attachment mechanism comprises a magnetic material disposed on at least one exterior surface of said pouch.

12. The absorbent article package as in claim 1, wherein said attachment mechanism comprises a hook-type material disposed on at least one exterior surface of said pouch.

13. The absorbent article package as in claim 1, wherein said attachment mechanism comprises a ribbon or string-like member attached to said pouch.

14. The absorbent article package as in claim 1, wherein said attachment mechanism comprises a loop configured on said pouch.

15. The absorbent article package as in claim 14, wherein said loop is formed of said wrapper material.

16. The absorbent article package as in claim 1, wherein said pouch is disguised as a women's accessory item.

17. The absorbent article as in claim 1, wherein said pouch and said absorbent article are flushable.

18. An individually wrapped feminine care interlabial absorbent article package, comprising:
an interlabial absorbent article;
a wrapper material configured into a closed pouch, said interlabial article disposed within said pouch and removable by a consumer opening said pouch and removing said interlabial article;
means disposed directly on an outer surface of said pouch for attaching said pouch at a discrete location.

19. The interlabial absorbent article package as set forth in claim 18, wherein said attaching means comprises a mechanical device.

20. The interlabial absorbent article package as set forth in claim 18, wherein said attaching means comprises an adhesive.
